(19) United States
(12) Patent Application Publication
(10) Pub. No.: US 2025/0271440 A1
(43) Pub. Date: Aug. 28, 2025

(54) CLINICAL TIER GRADING FOR PROGNOSIS OF IMMUNE CHECKPOINT INHIBITOR EFFICACY AND CLINICAL STATUS

(71) Applicant: Hays Documentation Specialists, LLC, San Mateo, CA (US)

(72) Inventor: Priya HAYS, San Mateo, CA (US)

(21) Appl. No.: 19/059,118

(22) Filed: Feb. 20, 2025

Related U.S. Application Data
(60) Provisional application No. 63/557,288, filed on Feb. 23, 2024.

(51) Int. Cl. G01N 33/574; C12Q 1/6886; G01N 33/68

(52) U.S. Cl. CPC G01N 33/57492; C12Q 1/6886; G01N 33/6872; C12Q 2600/118; G01N 2333/70521; G01N 2333/70578; G01N 2474/20

(57) ABSTRACT

Provided are methods and systems of clinical tier grading to assess prognosis of a treatment (e.g., an immune checkpoint inhibitor) efficacy and/or its safety.

### BACKGROUND

Clinical tier grading is used to stratify patients based on their symptoms and disease features. By utilizing a clinical grading system, patients can be grouped into different categories to assess management and treatment that is tailored to the patients. However, there is limited clinical tier grading proof-of-concepts developed to predict how a patient will respond to a treatment or the safety of a treatment, such as the use of an immune checkpoint inhibitor for treatment of cancer. Therefore, there exist a need to develop a clinical tier grading method and system for prognosis of treatment efficacy, such as immune checkpoint inhibitor efficacy and its safety in patients.

### SUMMARY

In one aspect, provided herein is a method comprising: a. isolating a sample from a subject; b. calculating a mRNA stemness index of the sample; and c. detecting an expression level of one or more checkpoint ligands, wherein the mRNA stemness index and the expression level of the one or more checkpoint ligands determine a prognosis of an efficacy and a side effect of an immune checkpoint inhibitor. In some embodiments, the sample is a tumor sample. In some embodiments, the method further comprises isolating a second sample from the subject, wherein the second sample is a normal tissue. In some embodiments, the subject has or is suspected to have a cancer. In some embodiments, the mRNA stemness index of at least 0.5 is a high-mRNA stemness index. In some embodiments, the mRNA stemness index of at most 0.5 is a low-mRNA stemness index. In some embodiments, the one or more checkpoint ligands comprises B7-1 (CD80), B7-2 (CD86), PD ligand 1 (PDL-1, CD274), MHC class II, Galectin, Poliovirus receptor, 4-1BBL, GITR ligand (GITRL), or derivatives thereof. In some embodiments, the expression level comprises a protein expression level or a gene expression level. In some embodiments, detecting the expression level comprises performing immunohistochemistry on the one or more checkpoint ligands. In some embodiments, the expression level of the one or more checkpoint ligands is decreased by at least about 2-fold, at least about 3-fold, at least about 4-fold, at least about 5-fold, at least about 10-fold, at least about 20-fold, at least about 30-fold, at least about 40-fold, at least about 50-fold, as compared to a control sample. In some embodiments, the expression level of the one or more checkpoint ligands is increased by at least about 2-fold, at least about 3-fold, at least about 4-fold, at least about 5-fold, at least about 10-fold, at least about 20-fold, at least about 30-fold, at least about 40-fold, at least about 50-fold, as compared to a control sample. In some embodiments, the method further comprises detecting an expression level of one or more immune checkpoint receptors. In some embodiments, the one or more immune checkpoint receptors comprise cytotoxic T lymphocyte antigen 4 (CTLA-4, CD152), programmed cell death protein 1 (PD-1, CD729), lymphocyte-activation 3 (LAG-3, CD223 T cell immunoglobulin mucin 3 (TIM-3), T cell immunoreceptor with Ig and ITIM domains (TIGIT), or derivatives thereof. In some embodiments, the method further comprises d) measuring tumor mutational burden, e) assessing risk level of the subject, or f) measuring an overall survival of the cancer. In some embodiments, the method further determines a tumor mutation burden category, a risk level category, or an overall survival prediction. In some embodiments, any one of steps a)-c) is repeated. In some embodiments, the immune checkpoint inhibitor comprises an anti-CTLA-4 inhibitor, an anti-PD-1 inhibitor, or an anti-PD-L1 inhibitor. In some embodiments, the anti-CTLA-4 inhibitor comprises ipilimumab: the anti-PD-1 inhibitor comprises nivolumab or pembrolizumab: or the anti-PD-L1 inhibitor comprises atezolizumab, avelumab, or durvalumab. In some embodiments, the side effects comprises cardiotoxicity, renal toxicity, neurological adverse events, dermatological reactions. In some embodiments, the efficacy is a low immune checkpoint inhibitor efficacy or a high immune checkpoint inhibitor efficacy.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the inventive concepts are set forth with particularity in the appended claims. A better understanding of the features and advantages of the present inventive concepts will be obtained by reference to the following detailed description that sets forth illustrative embodiments, in which the principles of the inventive concepts are utilized, and the accompanying drawings of which:

FIG. 1 shows an exemplary workflow for assessing the prognosis of a checkpoint inhibitor efficacy according to some embodiments herein.

FIG. 2 shows an exemplary workflow for assessing the prognosis of a checkpoint inhibitor efficacy and/or the prognosis of an immune checkpoint inhibitor side effects according to some embodiments herein.

FIG. 3 shows an exemplary workflow for assessing the prognosis of a checkpoint inhibitor efficacy and/or the prognosis of an immune checkpoint inhibitor side effects according to some embodiments herein.

FIG. 4 shows an exemplary workflow for assessing the prognosis of a checkpoint inhibitor efficacy and/or the prognosis of an immune checkpoint inhibitor side effects according to some embodiments herein.

### DETAILED DESCRIPTION

Disclosed herein are methods and systems of clinical tier grading to assess prognosis of a treatment (e.g., an immune checkpoint inhibitor) efficacy and/or its safety. The methods and systems comprise isolating sample from a subject, performing mRNA stemness index assay, performing analysis of protein and/or gene expression of the isolated sample, performing clinical characterization, and/or determining the prognosis of immune checkpoint inhibitor efficacy or safety as disclosed herein.

### I. SAMPLES

A sample can be obtained from a subject as part of any one of clinical tier grading methods or systems disclosed herein. A sample can be any biological sample isolated from a subject. In some embodiments, the sample is a tissue sample or cell free DNA. In some embodiments, the tissue sample can be a normal tissue sample. In some embodiments, the tissue sample or plasma sample can be a tumor sample. In some embodiments, the tumor sample can be from a immune desert region or immune rich region. In some embodiments, both the normal tissue or liquid biopsy and tumor sample can be obtained. In some embodiments, a normal tissue sample and a tumor sample can be obtained from the same subject. Liquid biopsy can also be obtained. In some embodiments, a normal tissue sample and a tumor sample can be obtained from different patients. A normal tissue or tumor sample can be obtained from a patient via various approaches, including, but not limited to, biopsy, needle aspirate, surgical incision, or other approaches thereof. In some embodiments, the sample (e.g., tissue sample) can be formalin-fixed and paraffin-embedded in blocks. In some embodiments, the sample can be frozen before analysis.

In some embodiments, the sample can be obtained from a healthy subject. In some embodiments, the sample can be obtained from a subject with a disease or disorder. In some embodiments, the sample can be obtained from a subject suspected from a disease or disorder. In some embodiments, the sample can be obtained before treatment (e.g., use of checkpoint inhibitors disclosed herein) of a subject with a disease or disorder. In some embodiments, the sample can be obtained after treatment (e.g., use of checkpoint inhibitors disclosed herein) of a subject with a disease or disorder. In some embodiments, the sample can be obtained before and after treatment (e.g., use of checkpoint inhibitors disclosed herein) of a subject with a disease or disorder. In some embodiments, the sample can be obtained from a subject during a treatment (e.g., use of checkpoint inhibitors disclosed herein) of a disease or disorder. In some embodiments, the sample can be obtained multiple times to monitor the effects of a treatment (e.g., use of checkpoint inhibitors disclosed herein) of a disease or disorder.

The methods and systems described herein can be applicable to a wide variety of disease or disorders, such as cancer. Examples of cancers include but not limited to solid tumors with high incidences such as melanoma, non-small cell lung cancer, breast cancer, colorectal cancer and non-immunogenic tumors such as prostate cancer.

### II. SAMPLE CHARACTERIZATION

The present disclosure provides methods or systems of clinical tier grading comprising characterization of a sample (e.g., healthy tissue or tumor sample) disclosed herein from a subject. In some embodiments, characterization of a sample can comprise calculating mRNAsi, measuring expression of one or more protein, measuring expression of one or more genes, measuring presence of one or more mutations, or combinations thereof.

A. mRNA Stemness Index (mRNAsi)

mRNA expression-based stemness index (mRNAsi) is an indicator of cancer stem cell characteristics of a sample. Cancer stem cell characteristics can comprise ability to self-renew, ability to constantly proliferate, and/or ability to differentiate into various cell types. mRNAsi of a sample can be calculated via machine learning (ML) model that uses the application of algorithms (e.g., supervised, semi-supervised, unsupervised, or reinforcement) on gene expression profiles or transcriptomics associated with the sample. In some embodiments, the machine learning model can be trained with data associated with cancer stem cell characteristics. Training the ML model can include, in some embodiments, selecting one or more untrained data models to train using a training data set. The selected untrained data models can include any type of untrained ML models for supervised, semi-supervised, self-supervised, or unsupervised machine learning. The selected untrained data models can be specified based upon input (e.g., user input) specifying relevant parameters to use as predicted variables or other variables to use as potential explanatory variables. For example, the selected untrained data models may be specified to generate an output (e.g., a prediction) based upon the input. Conditions for training the ML model from the selected untrained data models can likewise be selected, such as limits on the ML model complexity or limits on the ML model refinement past a certain point. The ML model can be trained (e.g., via a computer system such as a server) using the training data set. In some embodiments, a first subset of the training data set can be selected to train the ML model. The selected untrained data models may then be trained on the first subset of training data set using appropriate ML techniques, based upon the type of ML model selected and any conditions specified for training the ML model. In some cases, due to the processing power requirements of training the ML model, the selected untrained data models can be trained using additional computing resources (e.g., cloud computing resources). Such training can continue, in some embodiments, until at least one aspect of the ML model is validated and meets selection criteria to be used as a predictive model. In some embodiments, one or more aspects of the ML model can be validated using a second subset of the training data set (e.g., distinct from the first subset of the training data set) to determine accuracy and robustness of the ML model. Such validation may include applying the ML model to the second subset of the training data set to make predictions derived from the second subset of the training data. The ML model can then be evaluated to determine whether performance is sufficient based upon the derived predictions. The sufficiency criteria applied to the ML model can vary depending upon the size of the training data set available for training, the performance of previous iterations of trained models, or user-specified performance requirements. If the ML model does not achieve sufficient performance, additional training may be performed. Additional training can include refinement of the ML model or retraining on a different first subset of the training dataset, after which the new ML model may again be validated and assessed. When the ML model has achieved sufficient performance, in some cases, the ML can be stored for present or future use. The ML model can be stored as sets of parameter values or weights for analysis of further input (e.g., further relevant parameters to use as further predicted variables, further explanatory variables, further user interaction data, etc.), which can also include analysis logic or indications of model validity in some instances. In some embodiments, a plurality of ML models can be stored for generating predictions under different sets of input data conditions. In some embodiments, the ML model can be stored in a database (e.g., associated with a server).

In some embodiments, the mRNAsi can be calculated using one class logistic regression (OCLR) machine learning algorithm on gene expression profiles or transcriptomics associated with a sample. In some embodiments, gene expression profiles or transcriptomics can be obtained from public data. For example, omics data can be obtained from, but not limited to. The Cancer Genome Atlas (TCGA), MET500, Clinical Proteomic Tumor Analysis Consortium (CPTAC).

The values of mRNAsi range can range from 0 to 1. A sample disclosed herein with a mRNAsi values closer to 1 can represent a sample comprising more stem cell characteristics than a sample with a mRNAsi value closer to 0. In some embodiments, a mRNAsi value can be at least 0.1, at least 0.2, at least 0.3, at least 0.4, at least 0.5, at least 0.6, at least 0.7, at least 0.8, at least 0.9, or 1. In some embodiments, a mRNAsi value can be at most 0.1, at most 0.2, at most 0.3, at most 0.4, at most 0.5, at most 0.6, at most 0.7, at most 0.8, at most 0.9, or 1. In some embodiments, a mRNAsi value of at least 0.5 can be categorized as high-mRNAsi. In some embodiments, a mRNAsi value of at most 0.5 can be categorized as low-mRNAsi.

### B. Protein Expression

In some embodiments, a sample disclosed herein (e.g., tumor sample) can be analyzed for protein expression level of one or more immune checkpoint receptors or checkpoint ligands. For example, immune checkpoint receptors include, but not limited to, cytotoxic T lymphocyte antigen 4 (CTLA-4, CD152), programmed cell death protein 1 (PD-1, CD729), lymphocyte-activation 3 (LAG-3, CD223), T cell immunoglobulin mucin 3 (TIM-3), T cell immunoreceptor with Ig and ITIM domains (TIGIT), 4-1BB (CD137),). In some embodiments, the protein expression of PD-L1, PD-1, TIGIT, TIM-3, and LAG-3, or combinations thereof can be measured in a sample disclosed herein (e.g., tumor sample). In some embodiments, the protein expression of PD-L1 can be measured in a sample disclosed herein (e.g., tumor sample). In some embodiments, the protein expression of PD−1 can be measured in a sample disclosed herein (e.g., tumor sample) and a tumor proportion score can be measured. In some embodiments, the protein expression of TIGIT can be measured in a sample disclosed herein (e.g., tumor sample). In some embodiments, the protein expression of TIM-3 can be measured in a sample disclosed herein (e.g., tumor sample). In some embodiments, the protein expression of LAG-3 can be measured in a sample disclosed herein (e.g., tumor sample).

In some embodiments, protein level of any one of the immune checkpoint receptors or checkpoint ligands disclosed herein can be detected in a sample (e.g., tumor sample) using immunohistochemistry, enzyme-linked immunosorbent assays (ELISA), Western blot, immunoprecipitation, immunofluorescence, radioimmunoassay, dot blotting, immunodetection methods, mass spectrometry, HPLC or combinations thereof. In some embodiments, at least one, at least two, at least 3, at least 4, or more protein markers (e.g., immune checkpoint receptors or checkpoint ligands disclosed herein) can be measured in a sample. In some embodiments, the detected protein level of any one of the immune checkpoint receptors or checkpoint ligands disclosed herein in a sample is compared to a control sample (e.g., healthy tissue).

### C. Gene Expression and Mutation

In some embodiments, a sample disclosed herein (e.g., tumor sample) can be analyzed for biomarkers associated with a cancer type. In some embodiments, a sample disclosed herein (e.g., tumor sample) can exhibit high expression of genes that are lowly expressed in a control sample (e.g., healthy tissue) that is not associated with cancer. In some embodiments, a sample disclosed herein (e.g., tumor sample) can exhibit low expression of genes that are highly expressed in a control sample (e.g., healthy tissue) that is not associated with cancer.

In some embodiments, a sample disclosed herein (e.g., tumor sample) can be analyzed for gene expression or mRNA level of immune checkpoint receptors or checkpoint ligands disclosed herein. In some embodiments, the gene expression of PD-L1, PD-1, TIGIT, TIM-3, LAG-3, or combinations thereof can be measured in a sample disclosed herein (e.g., tumor sample). In some embodiments, the gene expression of PD-L1 can be measured in a sample disclosed herein (e.g., tumor sample). In some embodiments, the gene expression of PD-1 can be measured in a sample disclosed herein (e.g., tumor sample). In some embodiments, the gene expression of TIGIT can be measured in a sample disclosed herein (e.g., tumor sample). In some embodiments, the gene expression of TIM-3 can be measured in a sample disclosed herein (e.g., tumor sample). In some embodiments, the gene expression of LAG-3 can be measured in a sample disclosed herein (e.g., tumor sample).

In some embodiments, gene expression level (e.g., any one of the immune checkpoint receptors or checkpoint ligands disclosed herein) can be detected in a sample (e.g., tumor sample) using RNA-sequencing, bulk RNA-sequencing, qPCR, PCR, RT-PCR, multiplex qPCR, microarray analysis, fluorescence in situ hybridization, single-cell sequencing or combinations thereof. In some embodiments, at least one, at least two, at least 3, at least 4, or more gene markers (e.g., immune checkpoint receptors or checkpoint ligands disclosed herein) can be measured in a sample. In some embodiments, differential expression analysis (DESeq2) can be performed on transcriptome profiles from a sample disclosed herein (e.g., tumor sample) and a control sample (e.g., healthy sample) to generate a list of differentially expressed genes.

In some embodiments, presence of one or more mutations can be assessed for a sample (e.g., tumor sample) disclosed herein. In some embodiments, list of mutations from The Cancer Genome Atlas (TCGA) or other public database thereof can be generated to assess whether the mutations are present in a sample (e.g., tumor sample) disclosed herein. In some embodiments, the mutations present in a sample disclosed herein can be measured via methods such PCR, whole exome sequencing, targeted panel sequencing, next generation sequencing or combination thereof. In some embodiments, a tumor mutational burden (TMB) can be measured for a sample (e.g., tumor sample) to obtain the number of somatic mutations per megabase (muts/Mb) of interrogated genomic region. TMB can be ascertained by whole exome sequencing, targeted panel sequencing, next generation sequencing or combination thereof. In some embodiments, a sample disclosed herein (e.g., tumor sample) can have a TMB range of at least about 0-1 mut/Mb, at least about 1-1.5 mut/Mb, at least about 1.5-2 mut/Mb, at least about 2-2.5 mut/Mb, at least about 2.5-3 mut/Mb, at least about 3-3.5 mut/Mb, at least about 3.5-4 mut/Mb, at least about 4-4.5 mut/Mb, at least about 4.5-5 mut/Mb, at least about 5-10 mut/Mb, at least about 10-15 mut/Mb, at least about 15-20 mut/Mb, at least about 20-25 mut/Mb, at least about 25-30 mut/Mb, at least about 30-35 mut/Mb, at least about 35-40 mut/Mb, at least about 40-45 mut/Mb, at least about 45-50 mut/Mb or more. In some embodiments. TMB of less than or equal to 5 mut/Mb can be defined as low TMB. In some embodiments. TMB of more 5 and less than or equal to 20 mut/Mb can be defined as intermediate TMB. In some embodiments. TMB of more than 20 and less than or equal to 50 mut/Mb can be defined as high TMB. In some embodiments. TMB of more than 50 mut/Mb can be defined as very high TMB.

In some embodiments, a microsatellite instability can be measured for a sample (e.g., tumor sample) to obtain level of mutations in microsatellites. Microsatellite instability can be ascertained by whole exome sequencing, PCR, targeted panel sequencing, next generation sequencing or combination thereof. In some embodiments, the sample disclosed herein (e.g., tumor sample) can be classified into microsatellite instability-high, microsatellite instability-low, or microsatellite stable.

### D. Clinical Characterization

The present disclosure provides methods or systems of clinical tier grading comprising clinical characterization of a subject disclosed herein. In some embodiments, the clinical characterization of a subject can comprise subject's age, height, weight, ethnicity, or combinations thereof. In some embodiments, the clinical characterization can comprise analyzing medical history of a subject disclosed herein. In some embodiments, medical history of a subject disclosed herein can comprise allergies, current disease or disorders, previous disease or disorders, family health history, lifestyle choices (e.g., diet, smoking, exercise), occupation, medication, symptoms, or combinations thereof. In embodiments, clinical characterization of a subject disclosed herein can comprise analyzing outcome of diagnostic procedures. In some embodiments, diagnostic procedures can comprise laboratory tests, chest X-rays, EKG/EEG, urinalysis, physiological tests, non-cardiovascular imaging, diagnostic endoscopics, ultrasound/echocardiogram, skin biopsies, superficial needle biopsy, incisional biopsics, discography, cardiovascular imaging, or combinations thereof.

In some embodiments, the clinical characterization can further comprise determining a risk level of the disease (e.g., cancer) associated with a subject disclosed herein. In some embodiments, the clinical characterization can comprise determining a risk level of a subject disclosed herein based on the subject's clinical characterization. In some embodiments, the risk level of a disease or a subject disclosed herein can be categorized into minimal risk, low risk, moderate risk or high risk. In some embodiments, the clinical characterization can further comprise determining the overall survival with the disease (e.g., cancer) associated with a subject disclosed herein. In some embodiments, the overall survival with the disease can be calculated with a Kaplan-Meier survival analysis.

### III. CHECKPOINT INHIBITORS

The prognosis and safety of checkpoint inhibitors can be assessed with any one of the methods and systems of clinical tier grading disclosed herein. Checkpoint inhibitors are a type of immunotherapy, and often used for treatment for cancers. Checkpoint inhibitors can block checkpoint proteins (e.g., cytotoxic T lymphocyte associated protein 4 (CTLA-4), programmed cell death protein 1 (PD-1), or programmed cell death ligand 1 (PD-L1), lymphocyte-activation gene 3 (LAG-3) inhibitors) that prevents the immune system from attacking cancer cells. Examples of checkpoint inhibitors include, but not limited to, CTLA-4 inhibitors, PD-1 inhibitors, or PD-L1 inhibitors. Non-limiting examples of PD-1 inhibitors include nivolumab, pembrolizumab, Opdivo, or derivatives thereof. Non-limiting examples of CTLA-4 inhibitors include ipilimumab, Yervoy, or derivatives thereof. Non-limiting examples of PD-L1 inhibitors include atezolizumab, avelumab, durvalumab, Keytruda, or derivatives thereof. Non-limiting examples of LAG-3 inhibitors include relatlimab, or derivatives thereof. In some embodiments, one or more checkpoint inhibitors are administered to a subject.

In some embodiments, any one of the methods and systems of clinical tier grading disclosed herein can be used to determine the safety of checkpoint inhibitors. In some embodiments, the safety of checkpoint inhibitors can comprise immune-related adverse events (irAEs). In some embodiments, the safety of checkpoint inhibitors can comprise the presence of one or more side effects associated with checkpoint inhibitors (e.g., PD-1 inhibitors, CTLA-4 inhibitors, PD-L1 inhibitors, LAG-3 inhibitors). For example, side effects of checkpoint inhibitors include but are not limited to allergic reaction, flushing of the face, feeling dizzy, chills, tiredness, fatigue, loss of appetite, breathlessness, diarrhea, dry cough, dry skin, itchy skin, skin rash, nausea, constipation, muscle pain, or joint pain. In some embodiments, use of checkpoint inhibitors (e.g., PD-1 inhibitors, CTLA-4 inhibitors, PD-L1 inhibitors, LAG-3 inhibitors) can lead to autoimmune reactions, wherein the immune system can attack lungs, intestines, liver, endocrine glands, kidneys, or other organs.

### IV. METHODS AND SYSTEMS

Disclosed herein are methods and systems of clinical tier grading to assess prognosis of a treatment (e.g., an immune checkpoint inhibitor) efficacy and/or its safety (e.g., side effects). In some embodiments, the methods or systems can comprise measuring, in a sample obtained from a subject, 1) the mRNAsi level, 2) the levels of one or more immune checkpoint receptors or checkpoint ligands, 3) the tumor mutational burden, 4) risk level, and/or 5) overall survival. In some embodiments, measuring, in a sample obtained from a subject, the mRNAsi level, the levels of one or more checkpoint receptors or checkpoint ligands, the tumor mutational burden, risk level, and/or overall survival can be performed in any order. In some embodiments, measuring, in a sample obtained from a subject, the mRNAsi level, the levels of one or more checkpoint receptors or checkpoint ligands, the tumor mutational burden, risk level, and/or overall survival can be performed more than once. In some embodiments, the methods or systems can comprise measuring, in a sample obtained from a subject, 1) the mRNAsi level and 2) the levels of one or more checkpoint receptors or checkpoint ligands. In some embodiments, the mRNAsi level and/or the levels of one or more checkpoint receptors or checkpoint ligands can determine a tumor mutational burden category, a risk level category, and/or an overall survival.

In some embodiments, the mRNAsi can be calculated using one class logistic regression (OCLR) machine learning algorithm on gene expression profiles or transcriptomics associated with the sample. In some embodiments, gene expression profiles or transcriptomics can be obtained from public data disclosed herein. For example, omics data can be obtained from, but not limited to. The Cancer Genomc Atlas (TCGA), MET500, Clinical Proteomic Tumor Analysis Consortium (CPTAC). In some embodiments, high-mRNAsi determines a low immune checkpoint inhibitor efficacy or side effects. In some embodiments, low-mRNAsi determines a high immune checkpoint inhibitor efficacy or side effects.

In some embodiments, the levels of one or more immune checkpoint receptors or checkpoint ligands can be assessed by measuring the protein or gene expression level of the one or more checkpoint receptors or checkpoint ligands. In some embodiments, the one or more checkpoint receptors can be cytotoxic T lymphocyte antigen 4 (CTLA-4, CD152), programmed cell death protein 1 (PD-1, CD729), lymphocyte-activation 3 (LAG-3, CD223), T cell immunoglobulin mucin 3 (TIM-3), T cell immunoreceptor with Ig and ITIM domains (TIGIT), 4-1BB (CD137) or derivatives thereof. In some embodiments, the protein expression level of one or more checkpoint receptors or checkpoint ligands can be measured via immunohistochemistry, enzyme-linked immunosorbent assays (ELISA), Western blot, immunoprecipitation, immunofluorescence, radioimmunoassay, dot blotting, immunodetection methods, mass spectrometry, HPLC or combinations thereof. In some embodiments, the gene expression level of one or more checkpoint receptors or checkpoint ligands can be measured via RNA-sequencing, bulk RNA-sequencing, qPCR, PCR, RT-PCR, multiplex qPCR, microarray analysis, fluorescence in situ hybridization, single-cell sequencing or combinations thereof. In some embodiments, reduced expression of one or more immune checkpoint receptors or checkpoint ligands disclosed herein compared to a control sample (e.g., healthy tissue) determines a low immune checkpoint inhibitor efficacy or side effects. In some embodiments, increased expression of one or more immune checkpoint receptors or checkpoint ligands disclosed herein compared to a control sample (e.g., healthy tissue) determines a high immune checkpoint inhibitor efficacy or side effects.

In some embodiments, the tumor mutational burden (TMB) can be measured for a sample (e.g., tumor sample) to obtain the number of somatic mutations per megabase (muts/Mb) of interrogated genomic region. TMB can be ascertained by whole exome sequencing, targeted panel sequencing, next generation sequencing or combination thereof. In some embodiments. TMB of less than or equal to 5 mut/Mb can be defined as low TMB. In some embodiments. TMB of more 5 and less than or equal to 20 mut/Mb can be defined as intermediate TMB. In some embodiments. TMB of more than 20 and less than or equal to 50 mut/Mb can be defined as high TMB. In some embodiments. TMB of more than 50 mut/Mb can be defined as very high TMB. In some embodiments, a high TMB disclosed herein compared to a control sample (e.g., healthy tissue) determines a low immune checkpoint inhibitor efficacy or side effects. In some embodiments, a low TMB disclosed herein compared to a control sample (e.g., healthy tissue) determines a high immune checkpoint inhibitor efficacy or side effects.

In some embodiments, a microsatellite can further be measured as part of the methods and systems disclosed herein.

In some embodiments, methods and systems disclosed herein can comprise determining the clinical characterization of the subject disclosed herein. In some embodiments, the clinical characterization can comprise determining a risk level of a disease or a subject disclosed herein. In some embodiments, the risk level can be categorized into minimal risk, low risk, moderate risk or high risk. In some embodiments, minimal risk can comprise having a self-limited disease (e.g., cold). In some embodiments, moderate risk can comprise having one or more chronic disease with mild progression, two or more stable chronic diseases, undiagnosed disease with uncertain prognosis, acute diseases with systemic symptoms or complicated injuries, or combination thereof. In some embodiments, high risk can comprise having one or more chronic diseases with severe progression, diseases that can be life threatening.

In some embodiments, methods and systems disclosed herein can comprise determining the overall survival with the disease. In some embodiments, the overall survival with the disease can be calculated with a Kaplan-Meier survival analysis.

The methods or systems disclosed herein can also comprise additional biomarkers, clinical parameters, laboratory risk factors known to be present or associated with the clinical outcome of interest (e.g., prognosis of immune checkpoint inhibitor efficacy or safety), or combinations thereof. In some embodiments, one or more clinical parameters or risk factors can be incorporated into the methods or systems disclosed herein as an additional biomarker to assess efficacy of a treatment (e.g., use of immune checkpoint inhibitors) or as a pre-selection criterion to define a relevant type of sample to be analyzed. Non-limiting examples of clinical parameters or laboratory risk factors include tumor stage, tumor grade, tumor size, tumor location, tumor growth, lymph node status, histology, tumor thickness, proliferative index, tumor-infiltrating lymphocytes, proliferative index, or derivatives thereof.

In some embodiments, the method or system can further comprise grading or categorizing the sample into a tier that represents low or high prognosis of immune checkpoint inhibitor efficacy. In some embodiments, the method or system can further comprise grading or categorizing the sample into a tier that represents low or high prognosis of side effects for using immune checkpoint inhibitors. In some embodiments, the method or system can further comprise grading or categorizing the sample into tiers that represents low or high prognosis of immune checkpoint inhibitor efficacy and represents low or high prognosis of side effects for using immune checkpoint inhibitors. In some embodiments, the sample can be graded or categorized into a tier that represents low prognosis of immune checkpoint inhibitor efficacy and low prognosis of side effects for using immune checkpoint inhibitors. In some embodiments, the sample can be graded or categorized into a tier that represents high prognosis of immune checkpoint inhibitor efficacy and high prognosis of side effects for using immune checkpoint inhibitors. In some embodiments, the sample can be graded or categorized into a tier that represents low prognosis of immune checkpoint inhibitor efficacy and high prognosis of side effects for using immune checkpoint inhibitors. In some embodiments, the sample can be graded or categorized into a tier that represents high prognosis of immune checkpoint inhibitor efficacy and low prognosis of side effects for using immune checkpoint inhibitors. In some embodiments, grading or categorizing the sample can be expanded into more than two tiers. In some embodiments, the methods and systems of clinical tier grading can comprise two tiers, three tiers, four tiers, five tiers, or six tiers.

In some embodiments, the methods or systems disclosed herein can further be used to identify responders or non-responders to one or more immune checkpoint inhibitors disclosed herein. In some embodiments, the methods or systems disclosed herein can further be used to identify a subject who has complete response, partial response, stable disease or progressive disease to one or more immune checkpoint inhibitors disclosed herein. Complete response, partial response, stable disease or progressive disease can be defined by using the Response Evaluation Criteria in Solid Tumors (RECIST).

In some embodiments, the methods or systems disclosed herein can further be used to measure disease-free survival, progression-free survival, overall survival, or a combination thereof a subject disclosed herein. “Disease-free survival” refers to the length of time a subject is free from disease (e.g., cancer) after treatment. “Progression-free survival” refers to the length of time during and after treatment during which the disease being treated (e.g., cancer) does not get worse. Progression-free survival can include the amount of time a subject have experienced complete response, partial response, or stable disease. “Overall survival” refers to the percentage of subjects in a group who are likely to have survived after a particular duration of time.

### V. DEFINITIONS

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

As used in the specification and claims, the singular forms “a”, “an” and “the” include plural references unless the context clearly dictates otherwise. For example, the term “a sample” includes a plurality of samples, including mixtures thereof.

The terms “determining,” “measuring,” “evaluating,” “assessing,” “assaying,” and “analyzing” are often used interchangeably herein to refer to forms of measurement. The terms include determining if an element is present or not (for example, detection). These terms can include quantitative, qualitative or quantitative and qualitative determinations. Assessing can be relative or absolute, “Detecting the presence of” can include determining the amount of something present in addition to determining whether it is present or absent depending on the context.

The terms “subject,” “individual,” or “patient” are often used interchangeably herein. A “subject” can be a biological entity containing expressed genetic materials. The subject can be tissues, cells obtained in vivo. The subject can be a mammal. The mammal can be a human. The subject may be diagnosed or suspected of being at high risk for a disease. In some cases, the subject is not necessarily diagnosed or suspected of being at high risk for the disease.

As used herein, the term “about” a number refers to that number plus or minus 10% of that number. The term “about” a range refers to that range minus 10% of its lowest value and plus 10% of its greatest value.

As used herein, the terms “treatment” or “treating” are used in reference to a pharmaceutical or other intervention regimen for obtaining beneficial or desired results in the recipient. Beneficial or desired results include but are not limited to a therapeutic benefit and/or a prophylactic benefit. A therapeutic benefit can refer to eradication or amelioration of symptoms or of an underlying disorder being treated. Also, a therapeutic benefit can be achieved with the eradication or amelioration of one or more of the physiological symptoms associated with the underlying disorder such that an improvement is observed in the subject, notwithstanding that the subject may still be afflicted with the underlying disorder. A prophylactic effect includes delaying, preventing, or eliminating the appearance of a disease or condition, delaying or eliminating the onset of symptoms of a disease or condition, slowing, halting, or reversing the progression of a disease or condition, or any combination thereof. For prophylactic benefit, a subject at risk of developing a particular disease, or to a subject reporting one or more of the physiological symptoms of a disease may undergo treatment, even though a diagnosis of this disease may not have been made.

As used herein, the terms “machine learning (ML),” “machine learning techniques,” “machine learning operation,” and “machine learning model” generally refer to any system or analytical or statistical procedure that can progressively improve computer performance of a task. In some cases, ML can generally involve identifying and recognizing patterns in existing data in order to facilitate making predictions for subsequent data. ML can include a ML model (which may include, for example, a ML algorithm). Machine learning, whether analytical or statistical in nature, can provide deductive or abductive inference based on real or simulated data. The ML model can be a trained model. ML techniques can comprise one or more supervised, semi-supervised, self-supervised, or unsupervised ML techniques. For example, an ML model can be a trained model that is trained through supervised learning (e.g., various parameters are determined as weights or scaling factors). ML may comprise one or more of regression analysis, regularization, classification, dimensionality reduction, ensemble learning, meta learning, association rule learning, cluster analysis, anomaly detection, deep learning, or ultra-deep learning. ML may comprise, but is not limited to: k-means, k-means clustering, k-nearest neighbors, learning vector quantization, linear regression, non-linear regression, least squares regression, partial least squares regression, logistic regression, stepwise regression, multivariate adaptive regression splines, ridge regression, principal component regression, least absolute shrinkage and selection operation (LASSO), least angle regression, canonical correlation analysis, factor analysis, independent component analysis, linear discriminant analysis, multidimensional scaling, non-negative matrix factorization, principal components analysis, principal coordinates analysis, projection pursuit. Sammon mapping, t-distributed stochastic neighbor embedding, boosting, gradient boosting, bootstrap aggregation, ensemble averaging, decision trees, conditional decision trees, boosted decision trees, gradient boosted decision trees, random forests, stacked generalization, Bayesian networks, Bayesian belief networks, naïve Bayes, Gaussian naïve Bayes, multinomial naïve Bayes, hidden Markov models, hierarchical hidden Markov models, support vector machines, encoders, decoders, auto-encoders, stacked auto-encoders, perceptrons, multi-layer perceptrons, artificial neural networks, feedforward neural networks, convolutional neural networks, recurrent neural networks, long short-term memory, deep Boltzmann machines, deep convolutional neural networks, deep recurrent neural networks.

The section headings used herein are for organizational purposes only and are not to be construed as limiting the subject matter described.

### VI. EXAMPLES

The following examples are included for illustrative purposes only and are not intended to limit the scope of the inventive concepts.

### Example 1: Workflow for Determination of Immune Checkpoint Inhibitor Efficacy and Safety

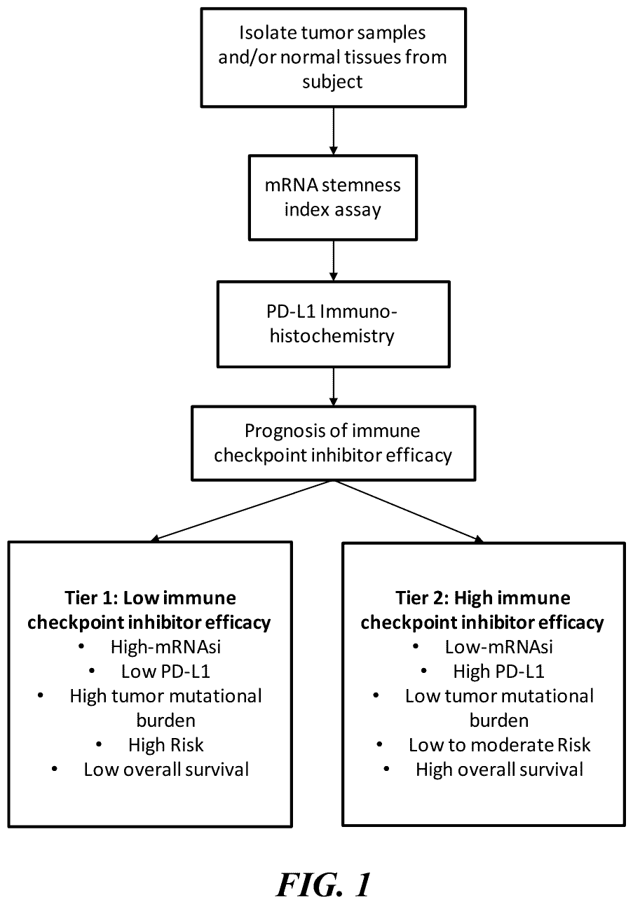

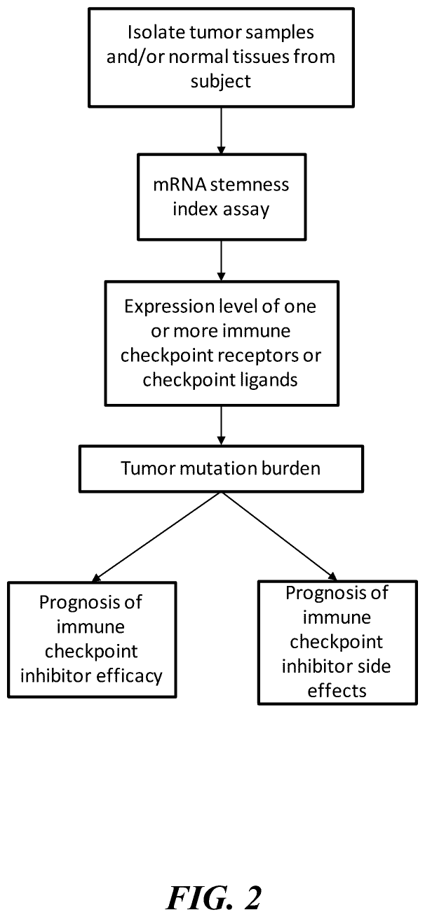

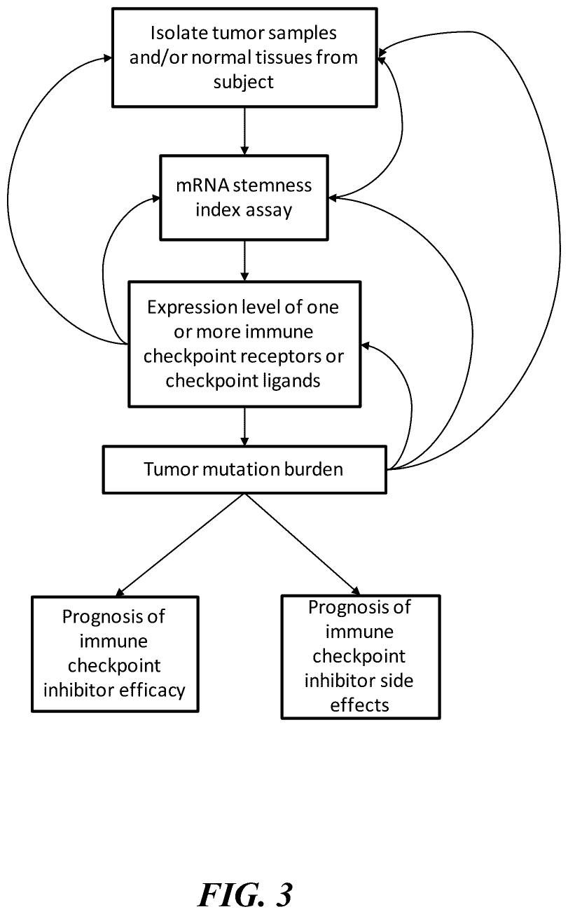

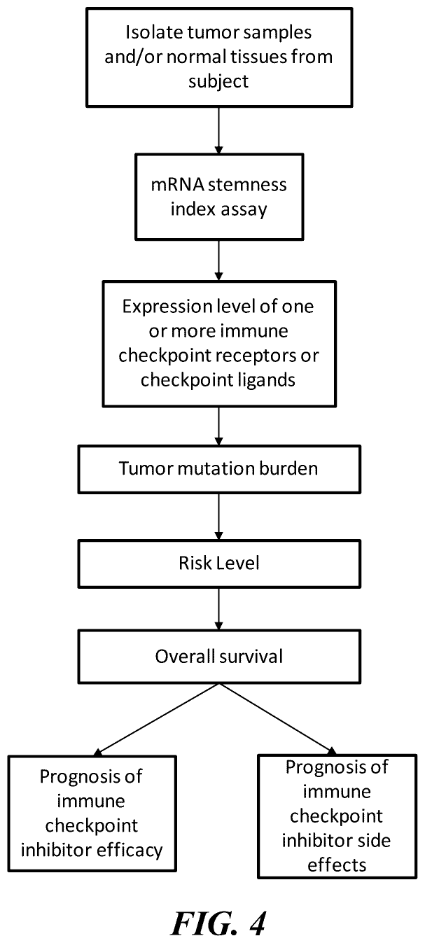

FIG. 1 shows an example of a workflow for determining an immune checkpoint inhibitor efficacy. The immune checkpoint inhibitor efficacy will be measured by first isolating tumor samples and normal tissues from a patient. Next, a mRNA stemness index assay will be performed on the samples to divide into low and high mRNAsi values. mRNAsi will identify differentially expressed genes between the tumor samples and normal samples. Furthermore, immunohistochemistry will be performed on samples isolated from the patient to identify low and high PD-L1 status. The outcome of the mRNAsi and PD-L1 expression will be used to categorize the patient into different tiers: a prognosis for low (Tier 1) or high (Tier 2) immune checkpoint inhibitor efficacy. For example, the signature of high-mRNAsi and low PD-L1 isolates a patient into high risk, low overall survival and high tumor burden category with a prognosis for low immune checkpoint inhibitor efficacy, while a signature of low-mRNAsi and high PD-L1 isolates a patient into low to moderate risk level patients, high overall survival and low mutational burden category with prognosis of high immune checkpoint inhibitor efficacy. As shown in FIG. 2, the workflow may also include measuring tumor mutation burden. Further, the measurement of mRNAsi, expression level of one or more immune checkpoint receptors or checkpoint ligands, and tumor mutation burden of a sample will lead to prognosis of an immune checkpoint inhibitor efficacy or prognosis of one or more side effects associated with an immune checkpoint inhibitor. As shown in FIG. 3, the different steps of the workflow may comprise of one or more feedback loops, where the outcome of the mRNAsi, expression level of one or more immune checkpoint inhibitors or checkpoint ligands, or tumor mutation burden will determine whether one or more steps will be repeated again to determine the prognosis of an immune checkpoint inhibitor or prognosis of one or more side effects associated with an immune checkpoint inhibitor. In addition, as shown in FIG. 4, the workflow may also comprise of other measurements such as risk level or overall survival of a disease or disorder of interest. The steps of the workflow may be modified to occur in different orders.

While some embodiments of the present inventive concepts have been shown and described herein, it will be obvious to those skilled in the art that such embodiments are provided by way of example only. Numerous variations, changes, and substitutions will now occur to those skilled in the art without departing from the inventive concepts. It should be understood that various alternatives to the embodiments of the inventive concepts described herein may be employed in practicing the inventive concepts. It is intended that the following claims define the scope of the inventive concepts and that methods and structures within the scope of these claims and their equivalents be covered thereby.

What is claimed is:

1. A method comprising:
a. isolating a sample from a subject;
b. calculating a mRNA stemness index of the sample; and
c. detecting an expression level of one or more checkpoint ligands,
wherein the mRNA stemness index and the expression level of the one or more checkpoint ligands determine a prognosis of an efficacy and a side effect of an immune checkpoint inhibitor.

2. The method of claim 1, wherein the sample is a tumor sample.

3. The method of claim 1, wherein the method further comprises isolating a second sample from the subject, wherein the second sample is a normal tissue.

4. The method of claim 1, wherein the subject has or is suspected to have a cancer.

5. The method of claim 1, wherein the mRNA stemness index of at least 0.5 is a high-mRNA stemness index.

6. The method of claim 1, wherein the mRNA stemness index of at most 0.5 is a low-mRNA stemness index.

7. The method of claim 1, wherein the one or more checkpoint ligands comprises PD ligand 1 or (PD-L1, CD274), or derivatives thereof.

8. The method of claim 1, wherein the expression level comprises a protein expression level or a gene expression level.

9. The method of claim 1, wherein detecting the expression level comprises performing immunohistochemistry on the one or more checkpoint ligands.

10. The method of claim 1, wherein the expression level of the one or more checkpoint ligands is decreased by at least about 2-fold, at least about 3-fold, at least about 4-fold, at least about 5-fold as compared to a control sample.

11. The method of claim 1, wherein the expression level of the one or more checkpoint ligands is increased by at least about 2-fold, at least about 3-fold, at least about 4-fold, at least about 5-fold, at least about as compared to a control sample.

12. The method of claim 1, wherein the method further comprises detecting an expression level of one or more immune checkpoint receptors.

13. The method of claim 12, wherein the one or more immune checkpoint receptors comprise cytotoxic T lymphocyte antigen 4 (CTLA-4, CD152), programmed cell death protein 1 (PD-1, CD729), lymphocyte-activation 3 (LAG-3, CD223), T cell immunoglobulin mucin 3 (TIM-3), T cell immunoreceptor with Ig and ITIM domains (TIGIT), 4-1BB (CD137) or derivatives thereof.

14. The method of claim 1, wherein the method further comprises d) measuring tumor mutational burden, e) assessing risk level of the subject, or f) measuring an overall survival of the cancer.

15. The method of claim 1, wherein the method further determines a tumor mutation burden category, a risk level category, or an overall survival prediction.

16. The method of claim 1, wherein any one of steps a)-c) is repeated.

17. The method of claim 1, wherein the immune checkpoint inhibitor comprises a CTLA-4 inhibitor, a PD-1 inhibitor, or a PD-L1 inhibitor.

18. The method of claim 17, wherein the CTLA-4 inhibitor comprises ipilimumab; the PD-1 inhibitor comprises nivolumab or pembrolizumab; or the PD-L1 inhibitor comprises atezolizumab, avelumab, or durvalumab.

19. The method of claim 1, wherein immune checkpoint inhibitors are associated with a number of side effects.

20. The method of claim 1, the efficacy is a low immune checkpoint inhibitor efficacy or a high checkpoint inhibitor efficacy.
